(19) United States
(12) Patent Application Publication
(10) Pub. No.: US 2005/0113913 A1
(43) Pub. Date: May 26, 2005

(54) Ocular implant for correcting vision problems

(75) Inventor: Jacques Duvert, Lyon (FR)

Correspondence Address: OLIFF & BERRIDGE, PLC, P.O. BOX 19928, ALEXANDRIA, VA, 22320, US

(73) Assignee: Jacques DUVERT, Lyon (FR)

(21) Appl. No.: 10/972,356

(22) Filed: Oct. 26, 2004

(30) Foreign Application Priority Data
Nov. 21, 2003 (FR) 03.13668

(51) Int. Cl. A61F 2/16

(52) U.S. Cl. 623/6.14; 623/6.22; 623/6.33; 623/6.34

(57) ABSTRACT

This ocular implant permits correction of various forms of ametropia. It comprises, on or within one and the same support (6) implantable in the eye (4), a first lens (9) which is fixed and defines an optic axis (A), and a second lens (11) which is movable between a position in which it is laterally offset from the first lens (9) and a position in which it is superposed on the first lens (9), along the same optic axis (A). One position is provided in particular for far vision, and the other position for near vision. The displacement of the second lens (11), between its two positions, can be controlled from the outside by magnetic means.

The present invention relates generally to ocular implants which are fitted in order to correct a number of common vision problems which are also designated by the term ametropia, for example astigmatism, hyperopia, myopia, presbyopia, and aphakia.

The traditional way of correcting vision problems is to wear corrective glasses. Some types of vision problems require bifocal or progressive lenses, which may cause the wearer some inconvenience. Generally speaking, the disadvantages of glasses are that they get scratched, require frequent cleaning, break and can be lost. Moreover, in the case of bifocal or progressive lenses intended to correct presbyopia, reading in a reclined position, for example in bed, is difficult because near vision is provided by the lower part of such lenses.

Traditional glasses are being increasingly superseded by contact lenses, or “corneal” lenses, which have recently undergone various developments and advances: flexible lenses, disposable lenses, progressive or multifocal lenses. However, contact lenses are poorly tolerated by some individuals. Use of progressive or multifocal lenses requires quite a long period of familiarization (cerebral learning), these lenses sometimes do not provide perfect near vision, and, finally, do not always do away with the occasional need to wear glasses.

Another recent area of research has been the application of various surgical techniques which involve reshaping the eye, in particular the cornea, so as to correct refractive errors of the eye. These surgical techniques include keratomileusis, epikeratophakia, radial keratotomy, LASIK (laser-assisted in situ keratomileusis) surgery, and the fitting of implants, especially in the form of an intracorneal lens, but this still remains an experimental area today. Such surgical techniques are still complex and risky and do not therefore guarantee a definite and perfect result, even subsequently after scar healing. They may give rise to complications such as the “haze” effect or may weaken the eye, with irreversible consequences. With this method, it is possible to operate differently on both eyes, in the case of presbyopia, by leaving one eye slightly myopic to allow it to focus for near vision, and giving the other eye normal far vision, but in this case too there is visual discomfort and a need for cerebral adaptation. As regards implants, these can be placed inside the eye either after ablation of the crystalline lens or behind the cornea without removal of the crystalline lens. These implants are generally monofocal, in which case they restore an excellent quality of vision, but the autonomy of near vision, like far vision, necessarily entails monovision. It has also been proposed to provide multifocal ocular implants which permit near vision and far vision but with a slightly lesser quality of vision and with the need for cerebral learning. Finally, known ocular implants do not permit correction of astigmatism.

The present invention aims to overcome all of the disadvantages set out above by providing a novel solution of the ocular implant type, avoiding all the problems currently posed by glasses, contact lenses, surgery and existing implants, the proposed solution permitting correction of all forms of ametropia, including astigmatism and, in particular, permitting far vision and near vision with a complete visual field.

To this end, the subject of the invention is an ocular implant for correcting vision problems, which principally comprises, on or within one and the same support implantable in an eye, a first lens or equivalent optical system which is fixed and defines an optic axis, and a second lens or equivalent optical system which is movable between a position in which it is laterally offset from the first lens and a position in which it is superposed on the first lens, along the same optic axis.

The subject of the invention, the ocular implant, thus has two functional positions resulting from the arrangement of its two lenses relative to one another and corresponding respectively to two focal distances:
in a first position, in which the second lens is offset from the axis of the first lens, vision will be through the first lens alone, this corresponding in particular to far vision;
in a second position, in which the second lens is superposed on the first lens, vision will be through the combination of both lenses, corresponding in particular to near vision.

It will be noted that the optic axis defined by the first, fixed lens coincides with the pupillary axis when the ocular implant is in place in the eye.

In a preferred embodiment of the invention, the second lens is mounted slidably in guides of the rail or track type, formed on the aforementioned support, so as to be displaceable by translation between its position offset from the first lens and its position superposed on the first lens.

Advantageously, the displacement of the second lens, between its position offset from the first lens and its position superposed on the first lens, is controllable, as required, from the outside by magnetic means. For example, the second lens comprises a metal filament inlaid in or surrounding this lens, while the means of external control is in the form of an object such as a ring worn or held by the user and equipped with a permanent magnet creating an external magnetic field acting on said filament. Thus, by suitably moving his finger, equipped with the suitable ring, in front of his eye, the user will be able to change this ocular implant from the “far vision” position to the “near vision” position, and vice versa.

The support of the two lenses is advantageously in the form of a cage or the like whose orifices allow the aqueous humor of the eye to pass inside. The cage or other, analogous support is provided with at least one haptic element, which can be produced in accordance with known principles, for holding the ocular implant in place.

This ocular implant can be made of various hydrophilic or hydrophobic materials, preferably those which are sufficiently flexible to ensure that the implant is pliable, which will in particular make it easier to fit in place. Thus, a flexible implant can be implanted by being passed through an incision of at most 3 millimetres formed centrally in the cornea, because otherwise there would be residual astigmatism after surgery. However, a flexible implant could become jammed if it is not correctly fitted, and it will be possible to avoid this risk by providing a category of rigid implants; in this case the incision will be made in the corneal limbus and may be greater than 3 millimetres in order to avoid any residual astigmatism after surgery.

The ocular implant of the invention can be fitted in the posterior chamber of the eye, behind the iris and in front of the crystalline lens, especially for correcting presbyopia (without cataract).

It should be noted that, in contrast to existing ocular implants, the implant of the present invention also makes it possible to correct astigmatism in far vision, and thus to correct all forms of ametropia or vision problems. This is achieved in particular with a cylindrical or spherocylindrical lens, which, for example, is the movable lens.

When operating on a cataract, the ocular implant of the invention can be placed in the crystalline capsule.

In all cases, the ocular implant of the invention makes it possible, for far vision and for near vision, to provide a complete visual field, in contrast to lenses of the bifocal or progressive type which permit good near vision only when the wearer looks downwards.

The invention will be better understood from the following description which is given with reference to the attached diagrammatic drawing showing, by way of example, an embodiment of this ocular implant for correcting vision problems:

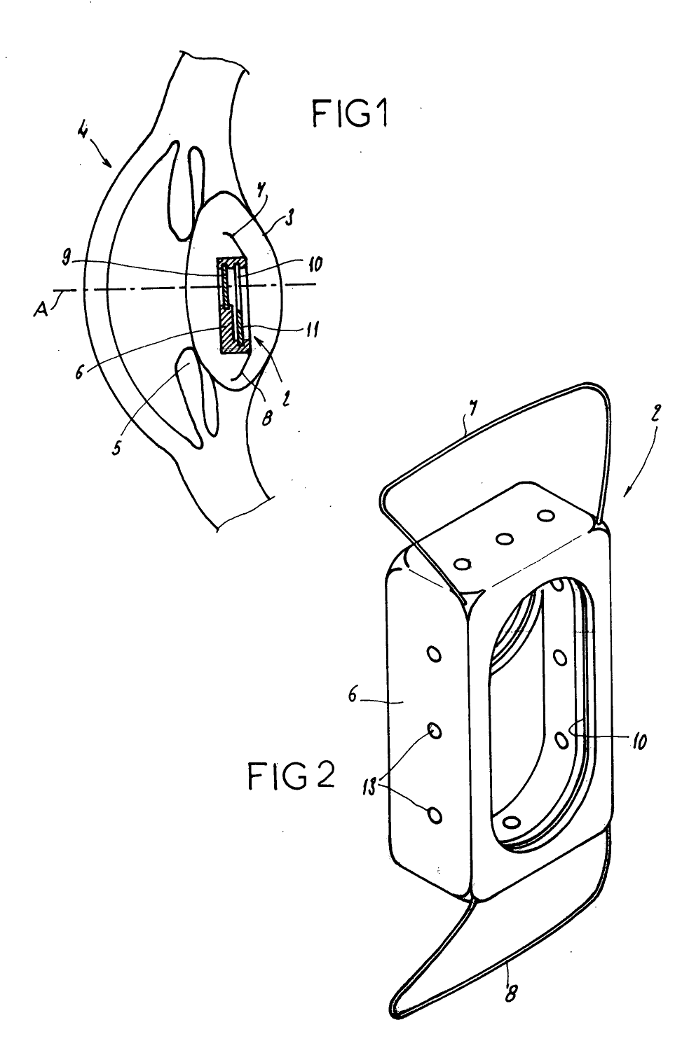

FIG. 1 shows a very diagrammatic cross section of an ocular implant according to the present invention, implanted in an eye;

FIG. 2 is a perspective view of this ocular implant;

FIG. 3 is a front view of the implant in a first position of use;

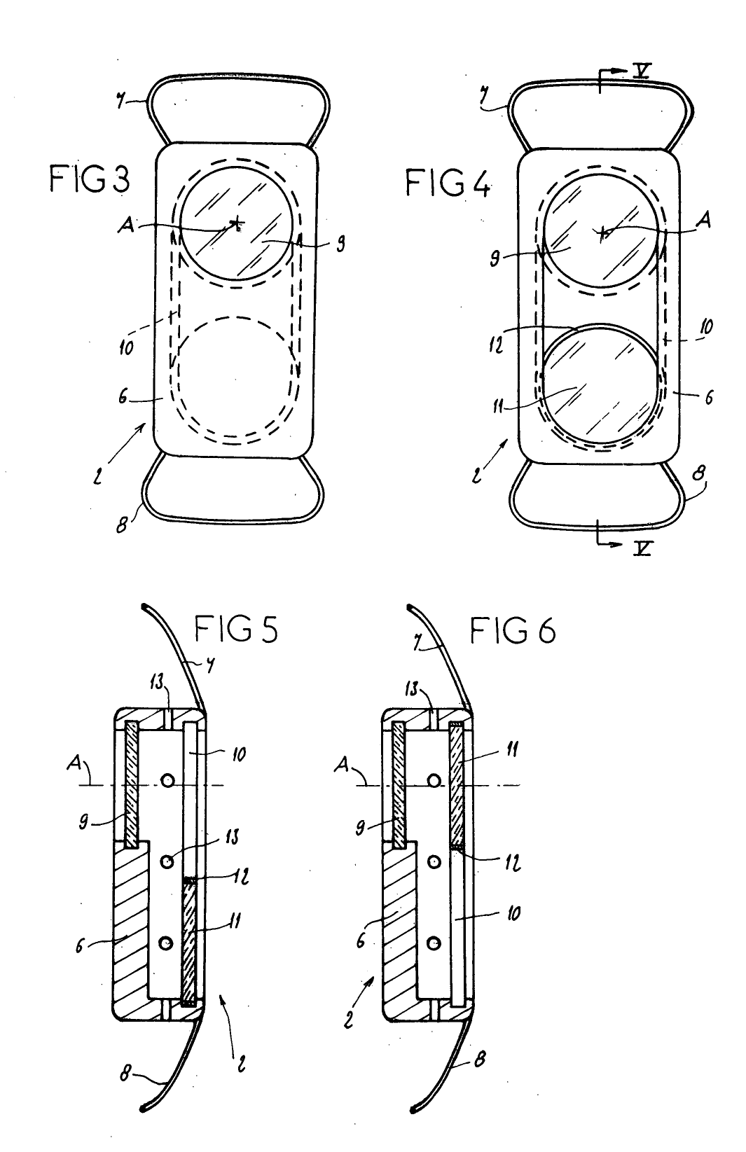

FIG. 4 is a rear view of the implant, in the same position;

FIG. 5 is a cross section of the implant along V-V in FIG. 4;

FIG. 6 is a cross section similar to FIG. 5, but illustrating a second position of use of the ocular implant.

FIG. 1 shows an ocular implant designated overall by reference number 2 and placed inside the crystalline capsule 3 of a human eye 4, behind the iris 5.

Referring also to FIGS. 2 to 6, the ocular implant 2 comprises a recessed support or “cage” 6 of oblong shape equipped on the outside with haptic elements 7, 8 for holding it in place.

Mounted on the support 6, in a fixed position, there is a first optic lens 9 which defines an optic axis A. When the ocular implant 2 is in place, this axis A coincides with the pupillary axis.

The support 6 forms, on the inside, two parallel lateral tracks 10 in which a second lens 11, omitted in FIG. 2, is mounted so as to slide. The second lens 11 can thus be displaced, in a plane parallel to that of the first lens 9, between a position laterally offset from the first lens 9 (FIGS. 3 to 5) and another position in which it is superposed on the first lens 9 (FIG. 6).

The second lens 11 is provided with a circular metal filament 12 inlaid in said lens or surrounding this lens 11, making it possible to move it alternately in translation from one position to the other, and vice versa, with the aid of an external magnetic field, for example produced by a ring equipped with a permanent magnet and worn on the user's finger. Thus, by moving his finger in a suitable way in front of his eye 4, the user can “switch” the ocular implant 2 from one position to the other.

In the first position (FIGS. 3 to 5), only the first lens 9 is present on the pupillary axis (optic axis A), the other lens 11 being laterally offset from it. The first lens 9 then provides, by itself, the appropriate correction for far vision.

In the other position (FIG. 6), the second lens 11 is superposed on the first lens 9, along the same optic axis A, and the combination of both lenses 9 and 11 then provides the appropriate correction for near vision, the second lens 12 being dimensioned accordingly.

Of course, the two lenses 9 and 11 are of a suitable type (convergent or divergent lenses) and of the focal distance appropriate to each case; they are represented here as cylindrical lenses of constant thickness purely for the purposes of illustration. For example, presbyopia is corrected at +3.5 diopters by the second lens 11.

The ocular implant 2 described above can be made of materials such as silicone, polymethylmethacrylate (PMMA), a copolymer of dimethylamide, polyvinyl, etc., and the material can be hydrophilic or hydrophobic and of greater or lesser flexibility. The use of an openwork “cage”, or of a support 6 provided with orifices 13, allows the aqueous humor of the eye 4 to pass through.

The thickness of the ocular implant 2 that is the subject of the invention will be a maximum of 3 millimetres, in particular for an implant intended to correct presbyopia, because in this case the implant will be placed between the crystalline lens and the iris, where it will have little space.

The scope of the invention, such as it is defined in the attached claims, would not be departed from:
by giving the ocular implant, in particular its support and its haptic elements, any desired shapes;
by making this ocular implant from any suitable materials which present no incompatibilities;
by reversing the roles of the two lenses, that is to say providing the fixed lens for near vision, and the movable lens (brought into line with the fixed lens) for far vision;
by using this ocular implant for correcting all types of ametropia: hyperopia, myopia, presbyopia, aphakia, possibly combined with astigmatism, the latter being corrected by adjusting the lenses of the implant in the corresponding axis, in the same way as the optician adjusts spectacle lenses on a frame;
by placing this implant in the eye in any suitable position, for example between the iris and the crystalline lens, the mode of implantation depending on the position sought, and it being possible to apply all operating techniques, preferably in such a way as to permit subsequent replacement of the ocular implant.

1. Ocular implant for correcting vision problems, or ametropia, characterized in that it comprises, on or within one and the same support implantable in an eye, a first lens or equivalent optical system which is fixed and defines an optic axis, and a second lens or equivalent optical system which is movable between a position in which it is laterally offset from the first lens and a position in which it is superposed on the first lens, along the same optic axis, the displacement of the second lens between the two positions being controllable from the outside.

2. Ocular implant according to claim 1, characterized in that the second lens is mounted slidably in guides of the rail or track type, formed on the support, so as to be displaceable by translation between its position offset from the first lens and its position superposed on the first lens.

3. Ocular implant according to claim 1, characterized in that the displacement of the second lens, between its position offset from the first lens and its position superposed on the first lens, is controllable from the outside by magnetic means.

4. Ocular implant according to claim 3, characterized in that the second lens comprises a metal filament inlaid in or surrounding this lens, while the means of external control is in the form of an object such as a ring worn or held by the user and equipped with a permanent magnet creating an external magnetic field acting on said filament.

5. Ocular implant according to claim 1, characterized in that the support of the two lenses is in the form of a cage or the like whose orifices allow the aqueous humor to pass inside.

6. Ocular implant according to claim 1, characterized in that it is produced using a flexible material, making this ocular implant 2 “pliable” in particular for its implantation.

7. Ocular implant according to claim 1, characterized in that the first position, in which the second lens is offset from the first lens, is provided for far vision, this therefore being effected by the first lens alone, whereas the second position, in which the second lens is superposed on the first lens, is provided for near vision, the wearer then looking through the combination of both lenses.

8. Ocular implant according to claim 2, characterized in that the displacement of the second lens, between its position offset from the first lens and its position superposed on the first lens, is controllable from the outside by magnetic means.

9. Ocular implant according to claim 2, characterized in that the support of the two lenses is in the form of a cage or the like whose orifices allow the aqueous humor to pass inside.

10. Ocular implant according to claim 3, characterized in that the support of the two lenses is in the form of a cage or the like whose orifices allow the aqueous humor to pass inside.

11. Ocular implant according to claim 4, characterized in that the support of the two lenses is in the form of a cage or the like whose orifices allow the aqueous humor to pass inside.

12. Ocular implant according to claim 2, characterized in that it is produced using a flexible material, making this ocular implant 2 “pliable” in particular for its implantation.

13. Ocular implant according to claim 3, characterized in that it is produced using a flexible material, making this ocular implant 2 “pliable” in particular for its implantation.

14. Ocular implant according to claim 4, characterized in that it is produced using a flexible material, making this ocular implant 2 “pliable” in particular for its implantation.

15. Ocular implant according to claim 5, characterized in that it is produced using a flexible material, making this ocular implant 2 “pliable” in particular for its implantation.

16. Ocular implant according to claim 2, characterized in that the first position, in which the second lens is offset from the first lens, is provided for far vision, this therefore being effected by the first lens alone, whereas the second position, in which the second lens is superposed on the first lens, is provided for near vision, the wearer then looking through the combination of both lenses.

17. Ocular implant according to claim 3, characterized in that the first position, in which the second lens is offset from the first lens, is provided for far vision, this therefore being effected by the first lens alone, whereas the second position, in which the second lens is superposed on the first lens, is provided for near vision, the wearer then looking through the combination of both lenses.

18. Ocular implant according to claim 4, characterized in that the first position, in which the second lens is offset from the first lens, is provided for far vision, this therefore being effected by the first lens alone, whereas the second position, in which the second lens is superposed on the first lens, is provided for near vision, the wearer then looking through the combination of both lenses.

19. Ocular implant according to claim 5, characterized in that the first position, in which the second lens is offset from the first lens, is provided for far vision, this therefore being effected by the first lens alone, whereas the second position, in which the second lens is superposed on the first lens, is provided for near vision, the wearer then looking through the combination of both lenses.

20. Ocular implant according to claim 6, characterized in that the first position, in which the second lens is offset from the first lens, is provided for far vision, this therefore being effected by the first lens alone, whereas the second position, in which the second lens is superposed on the first lens, is provided for near vision, the wearer then looking through the combination of both lenses.
